(19) United States
(12) Patent Application Publication
(10) Pub. No.: US 2020/0201183 A1
(43) Pub. Date: Jun. 25, 2020

(54) RESIST UNDERLAYER FILM FORMING COMPOSITION CONTAINING COMPOUND HAVING HYDANTOIN RING

(71) Applicant: NISSAN CHEMICAL CORPORATION, Tokyo (JP)

(72) Inventors: Hiroto OGATA, Toyama-shi (JP); Yuichi GOTO, Toyama-shi (JP); Masahisa ENDO, Toyama-shi (JP); Yuki USUI, Toyama-shi (JP); Takahiro KISHIOKA, Toyama-shi (JP)

(73) Assignee: NISSAN CHEMICAL CORPORATION, Tokyo (JP)

(21) Appl. No.: 16/317,939

(22) PCT Filed: Jun. 23, 2017
(86) PCT No.: PCT/JP2017/023211

§ 371 Date: Jan. 15, 2019

(30) Foreign Application Priority Data
Jul. 15, 2016 (JP) 2016-140315

(51) Int. Cl. G03F 7/11; C07D 403/14; G03F 7/004; G03F 7/16

(52) U.S. Cl. CPC G03F 7/11; G03F 7/168; G03F 7/0045; C07D 403/14

(57) ABSTRACT

A novel resist underlayer film forming composition containing a compound has a hydantoin ring. A resist underlayer film forming composition has a compound having at least two substituents of the following formula (1):

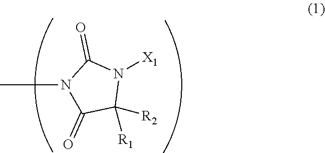

(wherein R₁ and R₂ are each independently a hydrogen atom or a methyl group, and X₁ is a C₁₋₃ hydroxyalkyl group or a C₂₋₆ alkyl group having one or two ether bonds in a main chain) in the molecule, and a solvent.

### TECHNICAL FIELD

The present invention relates to a resist underlayer film forming composition containing a compound having a hydantoin ring. Further, the present invention relates to a method for forming a photoresist pattern using the resist underlayer film forming composition.

### BACKGROUND ART

For example, a fine resist pattern formed on a substrate by a photolithography technique including an exposure step using a KrF excimer laser or an ArF excimer laser as a light source has been known in manufacturing a semiconductor element. Light of the KrF excimer laser or ArF excimer laser incident to a resist film before formation of a resist pattern (incident light) is reflected on a surface of the substrate to generate a standing wave in the resist film. This standing wave is known to prevent the formation of a resist pattern having a desired shape. For suppression of generation of the standing wave, an anti-reflective coating for absorbing incident light that is provided between the resist film and the substrate is also known. When this anti-reflective coating is provided under the resist film, the anti-reflective coating is required to exhibit a higher dry etching rate than that of the resist film.

Patent Documents 1 and 2 describe a compound for forming the anti-reflective coating. In particular, at least 95% of components in the composition described in Patent Document 2 have a molecular weight of less than 5,000 g/mol.

### PRIOR ART DOCUMENTS

### Patent Documents

Patent Document 1: International publication WO 2004/034148

Patent Document 2: International publication WO 2004/034435

### SUMMARY OF THE INVENTION

### Problem to be Solved by the Invention

In manufacturing a semiconductor element, a resist underlayer film satisfying all requirements including a high dry etching rate, a function as an anti-reflective coating during exposure, and filling a recess of a semiconductor substrate is required. A conventional resist underlayer film forming composition containing a low molecular weight compound can be embedded in a recess of a semiconductor substrate. However, the conventional resist underlayer film forming composition has high thermal sublimation properties. Therefore, when a resist underlayer film is formed, a sublimate is generated, and as a result, defects may be caused.

### Means for Solving the Problems

An aspect of the present invention is a resist underlayer film forming composition comprising a compound having at least two substituents of the following formula (1):

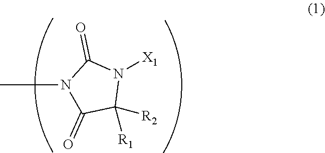

(wherein R₁ and R₂ are each independently a hydrogen atom or a methyl group, and X₁ is a C₁₋₃ hydroxyalkyl group or a C₂₋₆ alkyl group having one or two ether bonds in a main chain) in the molecule, and a solvent.

Examples of the C₁₋₃ hydroxyalkyl group include hydroxymethyl group, 1-hydroxyethyl group, 2-hydroxyethyl group, 1-hydroxypropyl group, 2-hydroxypropyl group, 3-hydroxypropyl group, 1-hydroxy-1-methylethyl group, and 2-hydroxy-1-methylethyl group. For example, the C₂₋₆ alkyl group having one or two ether bonds in a main chain is represented by a —R₄—OR₅— group. In this formula, R₄ is a C₁₋₃ alkylene group, and R₅ is a group defined by R₃ in formula (2) described below except for a hydrogen atom.

For example, the aforementioned compound is a compound of the following formula (2) having a weight average molecular weight of 300 to 5,000.

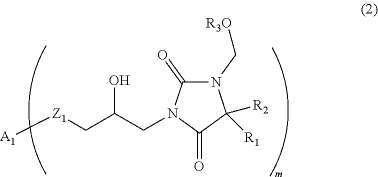

(wherein A₁ is a divalent to octavalent aliphatic group or a group having an aromatic or heterocyclic ring, Z₁ is a direct bond, an —O— group, or a —C(═O)O— group, R₁ and R₂ have the same definition as that in formula (1), R₃ is a hydrogen atom, a linear or branched alkyl group having a carbon atom number of 1 to 4, or a C₂₋₅ alkoxyalkyl group, and m is an integer of 2 to 8.)

Examples of the linear or branched alkyl group having a carbon atom number of 1 to 4 include methyl group, ethyl group, n-propyl group, isopropyl group, n-butyl group, sec-butyl group, isobutyl group, and tert-butyl group. Examples of the C₂₋₅ alkoxyalkyl group include methoxymethyl group, 1-methoxyethyl group, 2-methoxyethyl group, 1-methoxylpropyl group, 2-methoxylpropyl group, 3-methoxylpropyl group, 1-methoxy-1-methylethyl group, 2-methoxy-1-methylethyl group, ethoxymethyl group, 1-ethoxyethyl group, 2-ethoxyethyl group, 1-ethoxypropyl group, 2-ethoxypropyl group, 3-ethoxypropyl group, 1-ethoxy-1-methylethyl group, 2-ethoxy-1-methylethyl group, propoxymethyl group, 1-propoxyethyl group, 2-propoxyethyl group, 1-propoxy-1-methylethyl group, 2-propoxy-1-methylethyl group, isopropoxymethyl group, 1-isopropoxyethyl group, 2-isopropoxyethyl group, butoxymethyl group, sec-butoxymethyl group, isobutoxymethyl group, and tert-butoxymethyl group.

In the compound of formula (2), for example, m is an integer of 2 to 4, and A₁ is a divalent, trivalent, or tetravalent aliphatic group or a group having an aromatic or heterocyclic ring. Examples of the divalent, trivalent, or tetravalent aliphatic group or the group having an aromatic or heterocyclic ring include groups of the following formulae (a) to (v).

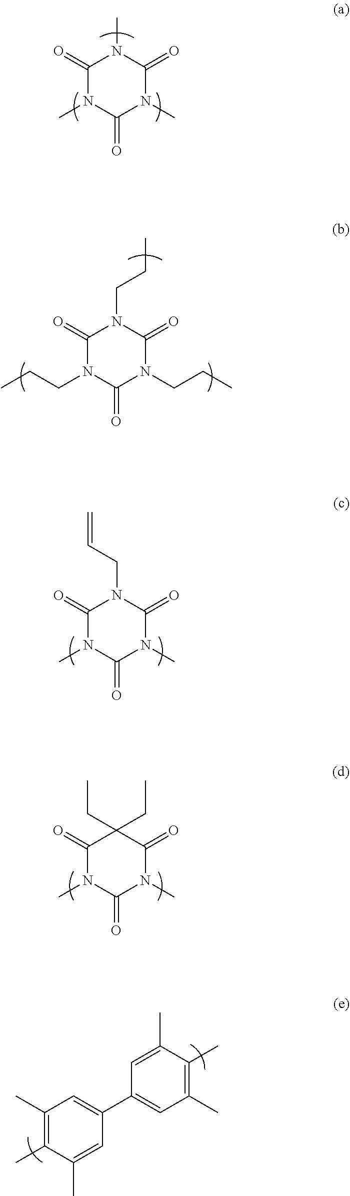

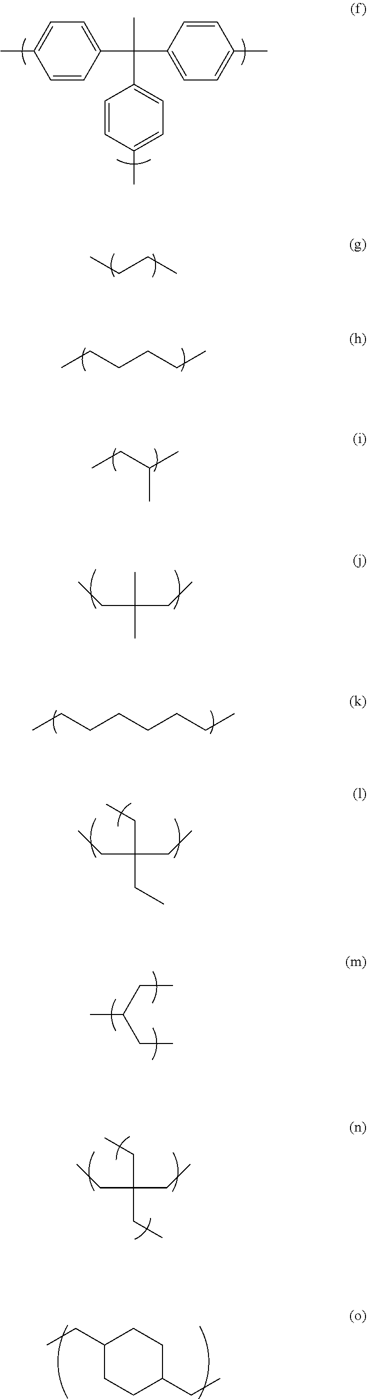

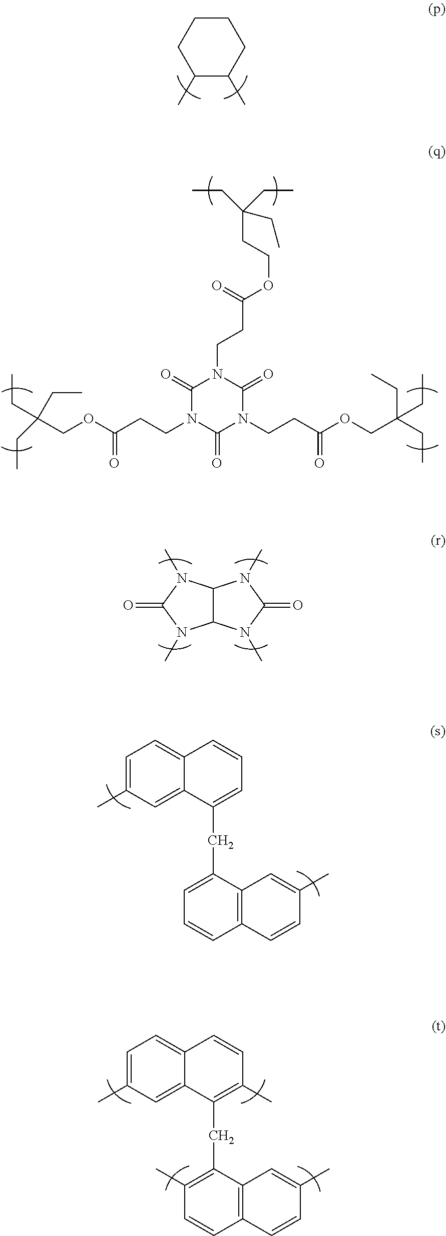

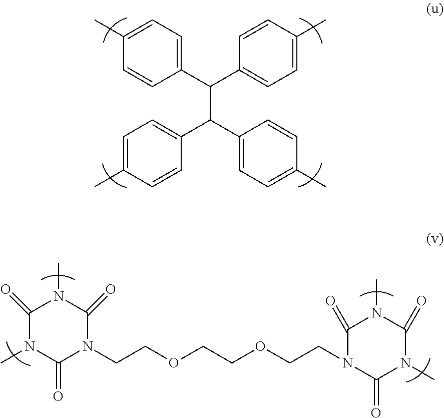

For example, the compound of formula (2) is a monomeric compound of the following formula (2a):

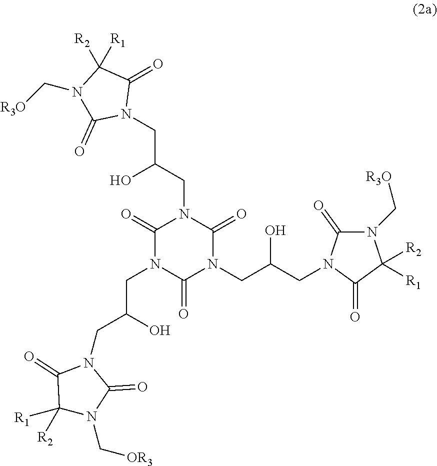

(wherein R₁ and R₂ have the same definition as that in formula (1), and R₃ has the same definition as that in formula (2)).

The resist underlayer film forming composition of the present invention may further contain a compound of the following formula (3), for example, in an amount of 1% by mass to 1,000% by mass relative to 100% by mass of the compound of formula (2).

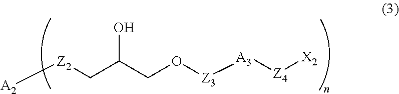

(wherein A₂ is a divalent to octavalent aliphatic group or a group having an aromatic or heterocyclic ring, Z₂ is a direct bond, an —)— group, or a —C(═O))— group, Z₃ and Z₄ are each independently a direct bond or a carbonyl group, A₃ is an arylene group in which at least one hydrogen atom is optionally substituted with hydroxyl group or halogeno group, or a C₁₋₃ alkylene group, X₂ is a hydroxyl group, a cyano group, or a C₁₋₆ alkyl group having one or two oxygen atoms in a main chain, and n is an integer of 2 to 8.)

Examples of the aliphatic group or the group having an aromatic or heterocyclic ring include the groups of formulae (a) to (v). Examples of the halogeno group include F group, Cl group, Br group, and I group. Examples of the arylene group include phenylene group and naphthylene group. For example, the C₁₋₆ alkyl group having one or two oxygen atoms in a main chain is represented by a —R₆—OR₇— group. In this formula, R₆ is a direct bond or a C₁₋₃ alkylene group, and R₇ is a group defined by R₃ in formula (2) described above except for a hydrogen atom.

The resist underlayer film forming composition of the present invention may further contain an additive selected from the group consisting of a cross-linking catalyst, a cross-linkable compound, and a surfactant. For example, the cross-linking catalyst is a thermal acid generator.

Another aspect of the present invention is a method for forming a photoresist pattern used in manufacturing a semiconductor device comprising steps of: applying the resist underlayer film forming composition to a semiconductor substrate having a hole or trench, and heating the semiconductor substrate at 150° C. to 350° C. to form a resist underlayer film; forming a photoresist layer on the resist underlayer film; exposing the semiconductor substrate coated with the resist underlayer film and the photoresist layer; and developing the photoresist layer after exposure.

### Effects of the Invention

From the resist underlayer film forming composition of the present invention, a resist underlayer film that satisfies all requirements including a high dry etching rate, a function as an anti-reflective coating during exposure, and filling a recess of a semiconductor substrate, and in which the amount of sublimate generated during baking is largely decreased is obtained. When the compound having at least two substituents of formula (1) described above in the molecule contained in the resist underlayer film forming composition of the present invention has a hydroxyalkyl group, the compound has self-crosslinking properties. Therefore, thermal sublimation properties are suppressed.

### BRIEF DESCRIPTION OF THE DRAWINGS

FIG. 1 is a schematic view illustrating a cross section of a SiO₂ wafer used in a test of embeddability in a trench (filling properties) of a resist underlayer film.

FIG. 2 is a cross-sectional SEM image of a SiO₂ wafer in which a trench is filled with a resist underlayer film formed from a resist underlayer film forming composition of Example 1.

FIG. 3 is a cross-sectional SEM image of a SiO₂ wafer in which a trench is filled with a resist underlayer film formed from a resist underlayer film forming composition of Example 2.

FIG. 4 is a cross-sectional SEM image of a SiO₂ wafer in which a trench is filled with a resist underlayer film formed from a resist underlayer film forming composition of Example 3.

FIG. 5 is a cross-sectional SEM image of a SiO₂ wafer in which a trench is filled with a resist underlayer film formed from a resist underlayer film forming composition of Example 4.

FIG. 6 is a cross-sectional SEM image of a SiO₂ wafer in which a trench is filled with a resist underlayer film formed from a resist underlayer film forming composition of Comparative Example 1.

### MODES FOR CARRYING OUT THE INVENTION

[Compound Having Hydantoin Ring]

The resist underlayer film forming composition of the present invention contains a compound having at least two substituents of formula (1) described above in the molecule. For example, the compound has a weight average molecular weight of 300 to 5,000, and preferably 500 to 3,000. It is preferable that the compound be a monomeric compound. Specific examples of the monomeric compound include compounds of the following formulae (2a-1) to (2a-4).

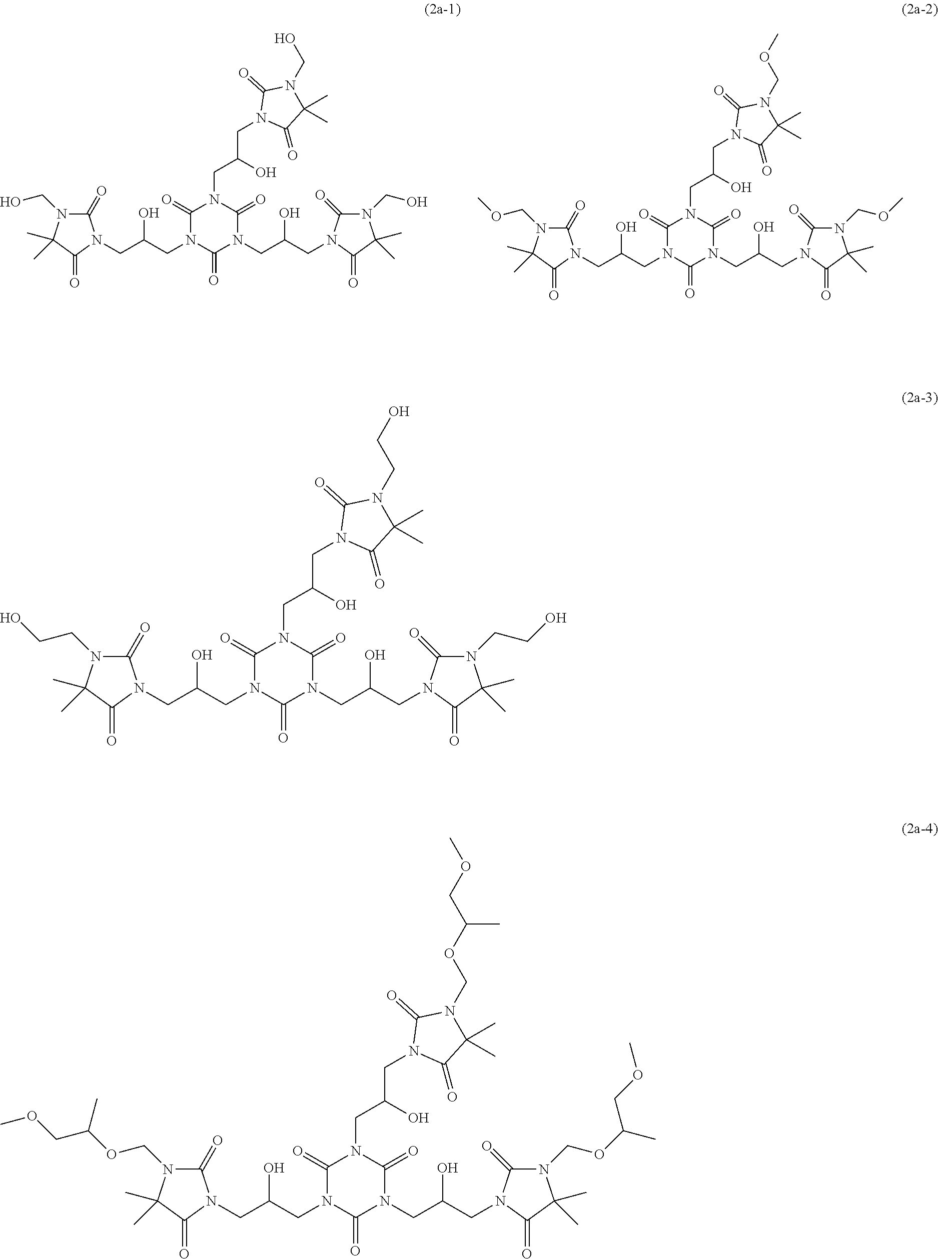

[Compound of Formula (3)]

The resist underlayer film forming composition of the present invention may further contain the compound of formula (3) described above. Specific examples of the compound of formula (3) include compounds of the following formulae (3a) to (3e).

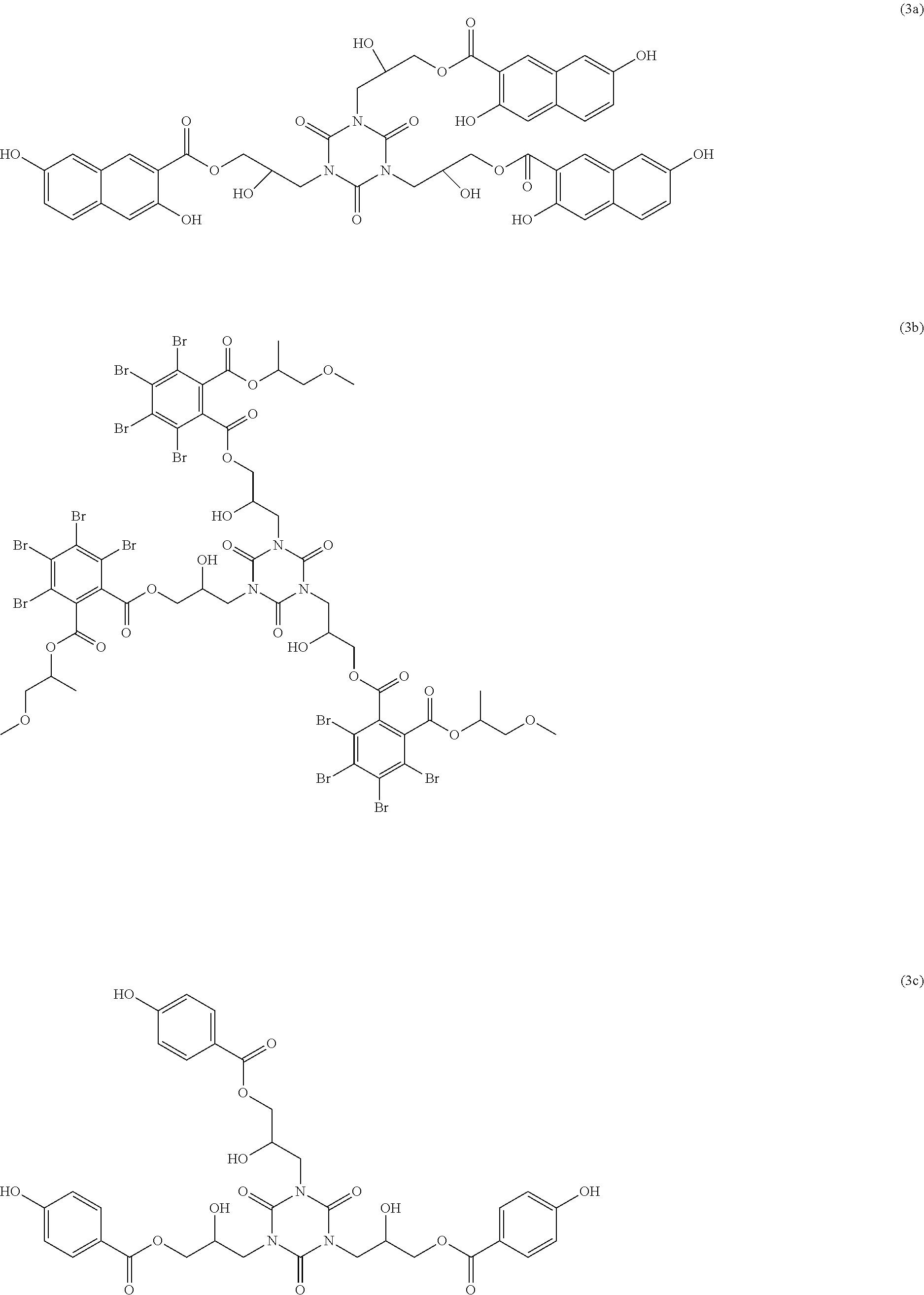

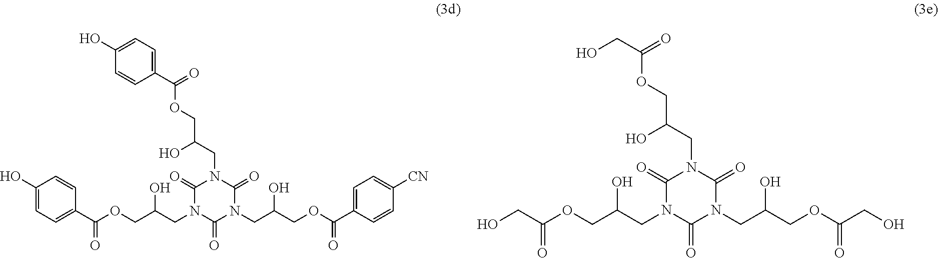 [SMILES: N#Cc1ccc(C(=O)OCC(O)Cn2c(=O)n(CC(O)COC(=O)c3ccc(O)cc3)c(=O)n(CC(O)COC(=O)c3ccc(O)cc3)c2=O)cc1 ; O=C(CO)OCC(O)Cn1c(=O)n(CC(O)COC(=O)CO)c(=O)n(CC(O)COC(=O)CO)c1=O]

When the compound of formula (3) is used, the content of the compound is 1% by mass to 1,000% by mass, and preferably 5% by mass to 500% by mass, relative to 100% by mass of the compound having at least two substituents of formula (1) in the molecule.

[Cross-linking Catalyst]

In order to promote a cross-linking reaction, the resist underlayer film forming composition of the present invention may contain a cross-linking catalyst in addition to the compound having at least two substituents of formula (1) described above in the molecule. As the cross-linking catalyst, for example, a sulfonic acid compound or a carboxylic acid compound, or a thermal acid generator may be used. Examples of the sulfonic acid compound include p-toluenesulfonic acid, pyridinium p-toluenesulfonic acid, 5-sulfosalicylic acid, 4-chlorobenzenesulfonic acid, 4-hydroxybenzenesulfonic acid, pyridinium phenolsulfonic acid, n-dodecyl benzenesulfonic acid, 4-nitrobenzenesulfonic acid, benzenedisulfonic acid, 1-naphthalenesulfonic acid, trifluoromethanesulfonic acid, and camphorsulfonic acid. Examples of the carboxylic acid compound include salicylic acid, citric acid, benzoic acid, and hydroxybenzoic acid. Examples of the thermal acid generator include K-PURE [registered trademark] CXC-1612, CXC-1614, TAG-2172, TAG-2179, TAG-2678, and TAG-2689 (available from King Industries, Inc.), and SI-45, SI-60, SI-80, SI-100, SI-110, and SI-150 (available from SANSHIN CHEMICAL INDUSTRY CO., LTD.).

One type of the cross-linking catalyst may be used, or two or more types thereof may be used in combination. When the cross-linking catalyst is used, the content of the cross-linking catalyst is 0.01% by mass to 10% by mass, and preferably 0.1% by mass to 5% by mass, relative to the content of the compound having at least two substituents of formula (1) in the molecule.

[Cross-Linkable Compound]

The resist underlayer film forming composition of the present invention may contain a cross-linkable compound to promote a cross-linking reaction. The cross-linkable compound is also called crosslinker. As the cross-linkable compound, a compound having at least two crosslink-forming substituents is preferably used. Examples thereof include a melamine-based compound, substituted urea-based compound, or aromatic compound that has at least two crosslink-forming substituents such as hydroxymethyl group and alkoxymethyl group, a compound having at least two epoxy groups, and a compound having at least two blocked isocyanate groups. Examples of the alkoxymethyl group include methoxymethyl group, 2-methoxyethoxymethyl group, and butoxymethyl group. As the cross-linkable compound, a nitrogen-containing compound having at least two, for example, two to four nitrogen atoms bonded to hydroxymethyl group or alkoxymethyl group is more preferably used. Examples of the nitrogen-containing compound include hexamethoxymethylmelamine, tetramethoxymethyl benzoguanamine,
1,3,4,6-tetrakis(methoxymethyl) glycoluril, 1,3,4,6-tetrakis(butoxymethyl) glycoluril,
1,3,4,6-tetrakis(hydroxymethyl) glycoluril, 1,3-bis(hydroxymethyl)urea,
1,1,3,3-tetrakis(butoxymethyl)urea, and 1,1,3,3-tetrakis(methoxymethyl)urea.

Examples of the aromatic compound having at least two hydroxymethyl groups or alkoxymethyl groups include 1-hydroxybenzene-2,4,6-trimethanol,
3,3′,5,5′-tetrakis(hydroxymethyl)-4,4′-dihydroxybiphenyl (trade name: TML-BP, available from Honshu Chemical Industry Co., Ltd.),
5,5′-[2,2,2-trifluoro-1-(trifluoromethyl)ethylidene]bis[2-hydroxy-1,3-benzenedimethanol] (trade name: TML-BPAF-MF, available from Honshu Chemical Industry Co., Ltd.),
2,2-dimethoxymethyl-4-t-butylphenol (trade name: DMOM-PTBP, available from Honshu Chemical Industry Co., Ltd.),
3,3′,5,5′-tetramethoxymethyl-4,4′-dihydroxybiphenyl (trade name: TMOM-BP, available from Honshu Chemical Industry Co., Ltd.),
bis(2-hydroxy-3-hydroxymethyl-5-methylphenyl)methane (trade name: DM-BIPC-F, available from Asahi Organic Chemicals Industry Co., Ltd.),
bis(4-hydroxy-3-hydroxymethyl-5-methylphenyl)methane (trade name: DM-BIOC-F, available from Asahi Organic Chemicals Industry Co., Ltd.), and
5,5′-(1-methylethylidene)bis(2-hydroxy-1,3-benzenedimethanol) (trade name: TM-BIP-A, available from Asahi Organic Chemicals Industry Co., Ltd.).

Examples of the compound having at least two epoxy groups include triglycidyl isocyanurate, 1,4-butanediol diglycidyl ether,
1,2-epoxy-4-(epoxyethyl)cyclohexane, glycerol triglycidyl ether, diethylene glycol diglycidyl ether, 2,6-diglycidyl phenyl glycidyl ether,
1,1,3-tris[p-(2,3-epoxypropoxy)phenyl]propane, 1,2-cyclohexanedicarboxylic acid diglycidyl ester, 4,4′-methylenebis(N,N-diglycidylaniline), 3,4-epoxycyclohexylmethyl
3,4-epoxycyclohexanecarboxylate, trimethylolethane triglycidyl ether,
bisphenol-A-diglycidyl ether, EPOLEAD [registered trademark] GT-401, GT-403, GT-301, and GT-302, and CELLOXIDE [registered trademark] 2021, and 3000 available from Daicel Corporation, 1001, 1002, 1003, 1004, 1007, 1009, 1010, 828, 807, 152, 154, 180S75, 871, and 872 available from Mitsubishi Chemical Corporation, EPPN201, 202, and EOCN-102, 103S, 104S, 1020, 1025, and 1027 available from NIPPON KAYAKU Co., Ltd., Denacol [registered trademark] EX-252, EX-611, EX-612, EX-614, EX-622, EX-411, EX-512, EX-522, EX-421, EX-313, EX-314, and EX-321 available from Nagase ChemteX Corporation, CY175, CY177, CY179, CY182, CY184, and CY192 available from BASF Japan Ltd., and EPICLON 200, 400, 7015, 835LV, and 850CRP available from DIC Corporation.

As the compound having at least two epoxy groups, a polymer compound may be used. The polymer compound can be used without particular limitation as long as it is a polymer having at least two epoxy groups. The polymer compound can be produced by addition polymerization of an addition-polymerizable monomer having epoxy group or a reaction of a polymer having hydroxy group with a compound having epoxy group such as epichlorohydrin and glycidyl tosylate. Examples of the polymer having at least two epoxy groups include addition polymerization polymers such as polyglycidyl acrylate, a copolymer of glycidyl methacrylate with ethyl methacrylate, and a copolymer of glycidyl methacrylate, styrene, and 2-hydroxyethyl methacrylate, and condensation polymerization polymers such as epoxy novolac. The weight average molecular weight of the polymer compound is for example 300 to 200,000. The weight average molecular weight is a value obtained by GPC using polystyrene as a standard sample.

As the compound having at least two epoxy groups, an epoxy resin having amino group may be further used. Examples of such an epoxy resin include YH-434 and YH-434L (all available from NSCC Epoxy Manufacturing Co., Ltd.).

Examples of the compound having at least two blocked isocyanate groups include TAKENATE [registered trademark] B-830 and B-870N available from Mitsui Chemicals, Inc., and VESTANAT [registered trademark]-B1358/100 available from Evonik Degussa GmbH. The compound may be used alone, or two or more types thereof may be used in combination.

When the cross-linkable compound is used, the content of the cross-linkable compound is 0.1% by mass to 80% by mass, and preferably 1% by mass to 60% by mass, relative to the content of the compound having at least two substituents of formula (1) in the molecule. When the content of the cross-linkable compound is too small or too large, the resistance of a film to be formed to a resist solvent may not be sufficiently achieved.

[Surfactant]

The resist underlayer film forming composition of the present invention may contain a surfactant to improve the application properties to a substrate. Examples of the surfactant include nonionic surfactants including polyoxyethylene alkyl ethers such as polyoxyethylene lauryl ether, polyoxyethylene stearyl ether, polyoxyethylene cetyl ether, and polyoxyethylene oleyl ether, polyoxyethylene alkylaryl ethers such as polyoxyethylene octylphenol ether and polyoxyethylene nonylphenol ether, polyoxyethylene-polyoxypropylene block copolymers, sorbitan fatty acid esters such as sorbitan monolaurate, sorbitan monopalmitate, sorbitan monostearate, sorbitan monooleate, sorbitan trioleate, and sorbitan tristearate, and polyoxyethylene sorbitan fatty acid esters such as polyoxyethylene sorbitan monolaurate, polyoxyethylene sorbitan monopalmitate, polyoxyethylene sorbitan monostearate, polyoxyethylene sorbitan trioleate, and polyoxyethylene sorbitan tristearate, fluorosurfactants including Eftop [registered trademark] EF301, EF303, and EF352 (available from Mitsubishi Materials Electronic Chemicals Co., Ltd.), MEGAFACE [registered trademark] F171, F173, R-30, R-30N, and R-40-LM (available from DIC Corporation), Fluorad FC430 and FC431 (available from Sumitomo 3M, Ltd.), and Asahi Guard (registered trademark) AG710, and Surflon [registered trademark] S-382, SC101, SC102, SC103, SC104, SC105, and SC106 (available from Asahi Glass Co., Ltd.), and organosiloxane polymer KP341 (available from Shin-Etsu Chemical Co., Ltd.). The surfactant may be added alone, or two or more types thereof may be added in combination.

When the surfactant is used, the content of the surfactant is for example 0.01% by mass to 5% by mass, and preferably 0.1% by mass to 3% by mass, relative to the content of the compound having at least two substituents of formula (1) in the molecule.

[Preparation of Composition]

The resist underlayer film forming composition of the present invention can be prepared by dissolving the aforementioned components in an appropriate solvent. The resist underlayer film forming composition can be used in a homogeneous solution state. Examples of such a solvent include ethylene glycol monomethyl ether, ethylene glycol monoethyl ether, methyl cellosolve acetate, ethyl cellosolve acetate, diethylene glycol monomethyl ether, diethylene glycol monoethyl ether, propylene glycol, propylene glycol monomethyl ether, propylene glycol monoethyl ether, propylene glycol monomethyl ether acetate, propylene glycol propyl ether acetate, toluene, xylene, methyl ethyl ketone, cyclopentanone, cyclohexanone, ethyl 2-hydroxypropionate, ethyl 2-hydroxy-2-methylpropionate, ethyl ethoxyacetate, ethyl hydroxyacetate, methyl 2-hydroxy-3-methylbutanoate, methyl 3-methoxypropinoate, ethyl 3-methoxypropionate, ethyl 3-ethoxypropionate, methyl 3-ethoxypropionate, methyl pyruvate, ethyl pyruvate, ethyl acetate, butyl acetate, ethyl lactate, butyl lactate, N,N-dimethylformamide, N,N-dimethylacetamide, and N-methylpyrrolidone may be used. The solvent may be used alone, or two or more types thereof may be used in combination. Further, a mixture of a solvent having a high boiling point such as propylene glycol monobutyl ether and propylene glycol monobutyl ether acetate with the aforementioned solvent may be used.

It is preferable that the prepared composition be used after filtration through a filter having a pore diameter of 0.2 μm, 0.1 μm, or 0.05 μm, for example. The resist underlayer film forming composition of the present invention has excellent storage stability at room temperature for an extended period of time.

Hereinafter, the use of the resist underlayer film forming composition of the present invention will be described. The composition of the present invention is applied to a substrate having a recess (e.g., a semiconductor substrate such as a silicon wafer and a germanium wafer that may be coated with a silicon oxide film, a silicon nitride film, or a silicon nitride oxide film) by an appropriate applying method such as a spinner and a coater. The substrate is then baked by a heating means such as a hot plate to form a resist underlayer film. A baking condition is appropriately selected from a baking temperature of 150° C. to 350° C. and a baking time of 0.3 minutes to 10 minutes. It is preferable that the baking temperature be 180° C. to 300° C. and the baking time be 0.5 minutes to 5 minutes. The resist underlayer film has a thickness of 0.005 μm to 3.0 μm, for example, 0.01 μm to 1.0 μm, or 0.05 μm to 0.5 μm.

When the temperature during baking is lower than the aforementioned range, cross-linking is insufficient, and intermixing between the resist underlayer film and a resist film to be formed as an upper layer may occur. When the temperature during baking is higher than the aforementioned range, intermixing between the resist underlayer film and the resist film may occur due to cleavage of cross-linking.

Subsequently, the resist film is formed on the resist underlayer film. The resist film can be formed by a general method, that is, by applying a photoresist solution to the resist underlayer film, followed by baking.

The photoresist solution used to form the resist film is not particularly limited as long as it can be sensitive to a light source used in exposure, and a negative photoresist solution or a positive photoresist solution may be used.

In order to form a resist pattern, exposure through a mask (reticle) for formation of a predetermined pattern is carried out. For example, a KrF excimer laser or an ArF excimer laser can be used for exposure. After exposure, post exposure bake is carried out, if necessary. A “post exposure bake” condition is appropriately selected from a heating temperature of 80° C. to 150° C. and a heating time of 0.3 minutes to 10 minutes. The resist pattern is then formed through a step of development with an alkaline developer.

Examples of the alkaline developer include alkaline aqueous solutions including an aqueous solution of an alkali metal hydroxide such as potassium hydroxide and sodium hydroxide, an aqueous solution of quaternary ammonium hydroxide such as tetramethylammonium hydroxide, tetraethylammonium hydroxide, and choline, and an aqueous solution of amine such as ethanolamine, propylamine, and ethylenediamine. Further, a surfactant or the like can be added to the developer. A development condition is appropriately selected from a development temperature of 5° C. to 50° C. and a development time of 10 seconds to 300 seconds.

### EXAMPLES

Hereinafter, specific examples of the resist underlayer film forming composition of the present invention will be described with reference to the following Synthesis Examples and Examples. The present invention is not limited to the Examples.

Apparatuses and the like used in measurement of weight average molecular weight of compounds obtained in the following Synthesis Examples are shown.
Apparatus: HLC-8320GPC manufactured by TOSOH CORPORATION
GPC column: KF-803L, KF-802, and KF-801 (manufactured by Showa Denko K.K.)
Column temperature: 40° C.
Solvent: tetrahydrofuran
Flow rate: 1.0 mL/min
Injection volume: 50 μL
Measurement time: 35 minutes
Standard sample: polystyrene (available from Showa Denko K.K.)
Detector: RI

### Synthesis Example 1

In a 300-mL reaction flask, 30.0 g of triglycidyl isocyanurate (available from Nissan Chemical Industries, Ltd.), 47.6 g of 1-hydroxymethyl-5,5-dimethylhydantoin (available from Tokyo Chemical Industry Co., Ltd.), 5.6 g of ethyltriphenylphosphonium bromide, and 194.2 g of ethanol were placed under a nitrogen atmosphere. This solution was heated and refluxed at 90° C., and a reaction was carried out for 24 hours. Subsequently, the ethanol was distilled off from the reaction solution by concentration. To the resultant, 355.5 g of propylene glycol monomethyl ether (hereinafter abbreviated as PGME) was added. To the mixture, 134.2 g of cation exchange resin (trade name: DOWEX [registered trademark] MONOSPHERE [registered trademark] 550A available from Muromachi Technos Co., Ltd.) and 134.2 g of anion exchange resin (trade name: AMBERLYST [registered trademark] 15JWET available from ORGANO CORPORATION) were added, and the mixture was stirred at 25° C. to 30° C. for 4 hours, and filtered. As a result, a solution containing a compound of the following formula was obtained. The obtained compound was analyzed by GPC. The weight average molecular weight in terms of standard polystyrene thereof was about 780.

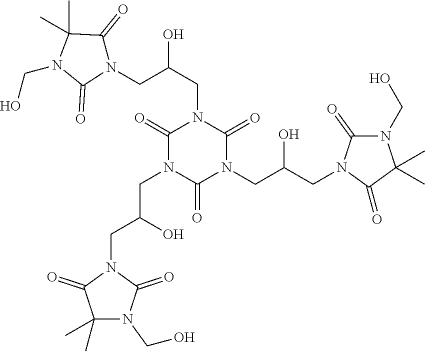 [SMILES: CC1(C)C(=O)N(CC(O)Cn2c(=O)n(CC(O)CN3C(=O)N(CO)C(C)(C)C3=O)c(=O)n(CC(O)CN3C(=O)N(CO)C(C)(C)C3=O)c2=O)C(=O)N1CO]

### Synthesis Example 2

In a 300-mL reaction flask, 15.0 g of triglycidyl isocyanurate (available from Nissan Chemical Industries, Ltd.), 30.8 g of 3,7-dihydroxynaphthalenecarboxylic acid (available from Midori Kagaku Co., Ltd.), 1.4 g of ethyltriphenylphosphonium bromide, and 109.9 g of PGME were placed under a nitrogen atmosphere. This solution was heated and refluxed at 140° C., and a reaction was carried out for 24 hours. To the reaction solution, 47.1 g of cation exchange resin (trade name: DOWEX [registered trademark] MONOSPHERE [registered trademark] 550A available from Muromachi Technos Co., Ltd.) and 47.1 g of anion exchange resin (trade name: AMBERLYST [registered trademark] 15JWET available from ORGANO CORPORATION) were added, and the mixture was stirred at 25° C. to 30° C. for 4 hours, and filtered. As a result, a solution containing a compound of the following formula was obtained. The obtained compound was analyzed by GPC. The weight average molecular weight in terms of standard polystyrene thereof was about 1,000.

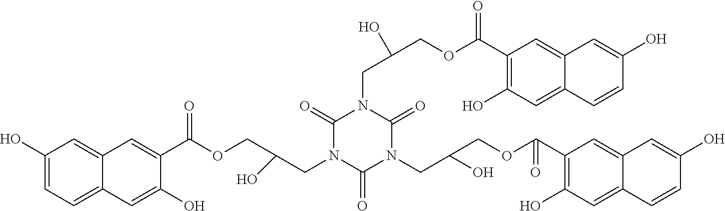 [SMILES: O=C(OCC(O)Cn1c(=O)n(CC(O)COC(=O)c2cc3cc(O)ccc3cc2O)c(=O)n(CC(O)COC(=O)c2cc3cc(O)ccc3cc2O)c1=O)c1cc2cc(O)ccc2cc1O]

### Synthesis Example 3

In a 100-mL reaction flask, 2.5 g of triglycidyl isocyanurate (available from Nissan Chemical Industries, Ltd.), 11.6 g of tetrabromophthalic anhydride (available from Tokyo Chemical Industry Co., Ltd.), 0.2 g of ethyltriphenylphosphonium bromide, and 33.5 g of PGME were placed under a nitrogen atmosphere. This solution was heated and refluxed at 140° C., and a reaction was carried out for 24 hours. To the reaction solution, 14.4 g of cation exchange resin (trade name: DOWEX [registered trademark] MONOSPHERE [registered trademark] 550A available from Muromachi Technos Co., Ltd.) and 14.4 g of anion exchange resin (trade name: AMBERLYST [registered trademark] 15JWET available from ORGANO CORPORATION) were added, and the mixture was stirred at 25° C. to 30° C. for 4 hours, and filtered. As a result, a solution containing a compound of the following formula was obtained. The obtained compound was analyzed by GPC. The weight average molecular weight in terms of standard polystyrene thereof was about 1,500.

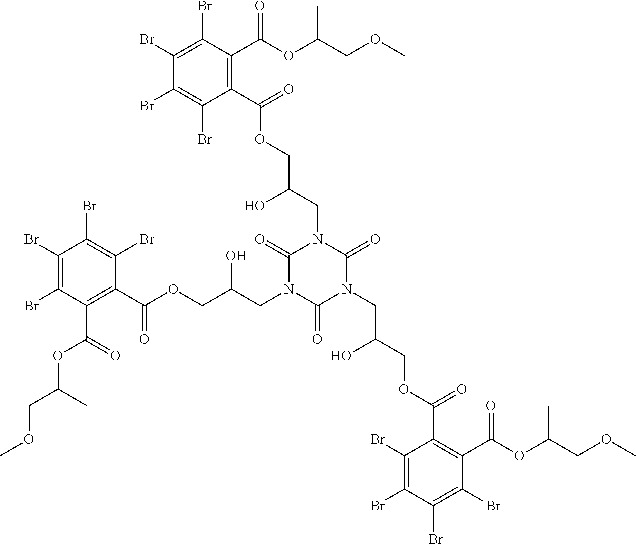

### Example 1

In 4.45 g of the solution containing 0.66 g of the compound obtained in Synthesis Example 1 (a solvent was PGME used in synthesis), 0.016 g of 4-hydroxybenzenesulfonic acid (available from Tokyo Chemical Industry Co., Ltd.), 9.10 g of PGME, and 1.43 g of propylene glycol monomethyl ether acetate (hereinafter abbreviated as PGMEA) were mixed to obtain a solution containing 4.51% by mass of mixed components except for the solvent. The solution was filtered through a microfilter made of polytetrafluoroethylene having a pore diameter of 0.2 μm to prepare a resist underlayer film forming composition.

### Example 2

In 3.42 g of the solution containing 0.51 g of the compound obtained in Synthesis Example 1 (a solvent was PGME used in synthesis), 0.017 g of 5-sulfosalicylic acid (available from Tokyo Chemical Industry Co., Ltd.), 0.63 g of the solution containing 0.18 g of the compound obtained in Synthesis Example 2 (a solvent was PGME used in synthesis), 0.00051 g of surfactant (trade name: R-30N available from DIC Corporation), 14.06 g of PGME, and 1.88 g of PGMEA were mixed to obtain a solution containing 3.54% by mass of mixed components except for the solvent. The solution was filtered through a microfilter made of polytetrafluoroethylene having a pore diameter of 0.2 μm to prepare a resist underlayer film forming composition.

### Example 3

In 59.42 g of the solution containing 8.84 g of the compound obtained in Synthesis Example 1 (a solvent was PGME used in synthesis), 0.44 g of 1,3,4,6-tetrakis(methoxymethyl) glycoluril (trade name: Powderlink 1174 available from Mitsui Cytec Ltd.), 0.22 g of 5-sulfosalicylic acid (available from Tokyo Chemical Industry Co., Ltd.), 10.94 g of the solution containing 3.09 g of the compound obtained in Synthesis Example 2 (a solvent was PGME used in synthesis), 0.0088 g of surfactant (trade name: R-30N available from DIC Corporation), 245.22 g of PGME, and 33.74 g of PGMEA were mixed to obtain a solution containing 3.60% by mass of mixed components except for the solvent. The solution was filtered through a microfilter made of polytetrafluoroethylene having a pore diameter of 0.2 μm to prepare a resist underlayer film forming composition.

### Example 4

In 105.65 g of the solution containing 15.60 g of the compound obtained in Synthesis Example 1 (a solvent was PGME used in synthesis), 1.09 g of 1,3,4,6-tetrakis(methoxymethyl) glycoluril (trade name: Powderlink 1174 available from Mitsui Cytec Ltd.), 0.039 g of pyridinium phenolsulfonic acid (available from Midori Kagaku Co., Ltd.), 19.33 g of the solution containing 5.46 g of the compound obtained in Synthesis Example 2 (a solvent was PGME used in synthesis), 0.016 g of surfactant (trade name: R-30N available from DIC Corporation), 431.40 g of PGME, and 59.48 g of PGMEA were mixed to obtain a solution containing 3.60% by mass of mixed components except for the solvent. The solution was filtered through a microfilter made of polytetrafluoroethylene having a pore diameter of 0.2 μm to prepare a resist underlayer film forming composition.

### Comparative Example 1

In 5.67 g of the solution containing 1.56 g of the compound obtained in Synthesis Example 2 (a solvent was PGME used in synthesis), 0.39 g of 1,3,4,6-tetrakis(methoxymethyl) glycoluril (trade name: Powderlink 1174 available from Mitsui Cytec Ltd.), 0.039 g of pyridinium p-toluenesulfonic acid (available from Tokyo Chemical Industry Co., Ltd.), 0.0078 g of surfactant (trade name: R-30N available from DIC Corporation), 34.29 g of PGME, and 9.60 g of PGMEA were mixed to obtain a solution containing 4.00% by mass of mixed components except for the solvent. The solution was filtered through a microfilter made of polytetrafluoroethylene having a pore diameter of 0.2 μm to prepare a resist underlayer film forming composition.

### Comparative Example 2

In 38.26 g of the solution containing 10.13 g of the compound obtained in Synthesis Example 3 (a solvent was PGME used in synthesis), 1.69 g of 1,3,4,6-tetrakis(methoxymethyl) glycoluril (trade name: Powderlink 1174 available from Mitsui Cytec Ltd.), 0.084 g of pyridinium p-toluenesulfonic acid (available from Tokyo Chemical Industry Co., Ltd.), 19.30 g of PGME, and 110.67 g of PGMEA were mixed to obtain a solution containing 7.00% by mass of mixed components except for the solvent. The solution was filtered through a microfilter made of polytetrafluoroethylene having a pore diameter of 0.2 μm to prepare a resist underlayer film-forming composition.

[Elution Test into Photoresist Solvent]

The resist underlayer film forming composition prepared in each of Examples 1 to 4 and Comparative Examples 1 and 2 was applied to a silicon wafer by a spinner, and then baked on a hot plate at 215° C. for one minute to form each resist underlayer film (thickness: 0.1 μm). The resist underlayer films were each immersed in PGME and PGMEA that were solvents used for a photoresist solution. It was confirmed that the resist underlayer films were insoluble in both the solvents.

[Test of Optical Parameter]

The resist underlayer film forming composition prepared in each of Examples 1 to 4 and Comparative Examples 1 and 2 was applied to a silicon wafer by a spinner, and then baked on a hot plate at 215° C. for one minute to form each resist underlayer film (thickness: 0.1 μm). The refractive index (n value) and extinction coefficient (k value) of the resist underlayer films at wavelengths of 193 nm and 248 nm were measured by an ellipsometer (VUV-VASEVU-302 manufactured by J. A. Woollam Co.). The results are shown in Table 1. It is desirable that the resist underlayer films have a k value of 0.1 or more at wavelengths of 193 nm and 248 nm to achieve a sufficient anti-reflection function.

[Measurement of Dry Etching Rate]

A resist underlayer film was formed on a silicon wafer by the same method as described above using the resist underlayer film forming composition in each of Examples 1 to 4 and Comparative Examples 1 and 2. The dry etching rate of the resist underlayer films was measured under a condition of using a RIE system manufactured by SAMCO INC., and using N₂ as a dry etching gas. A photoresist solution (trade name: V146G available from JSR Corporation) was applied to a silicon wafer by a spinner, and baked on a hot plate at 110° C. for one minute to form a photoresist film. The dry etching rate of the photoresist film was measured under a condition of using a RIE system manufactured by SAMCO INC., and using N₂ as a dry etching gas. The dry etching rate of each of the resist underlayer films was calculated when the dry etching rate of the photoresist film was 1.00. The results are shown as “selection ratio” in Table 1 described blow.

[Measurement of Sublimate Amount]

A silicon wafer having a diameter of 4 inches was coated with the resist underlayer film forming composition in each of Examples 1 to 4 and Comparative Examples 1 and 2 at 1,500 rpm for 60 seconds by a spin coating method. The silicon wafer was set in a sublimate amount measurement apparatus (see International publication WO2007/111147) integrated with a hot plate, and baked for 120 seconds. The sublimate was collected by a quartz crystal microbalance (QCM) sensor, that is, a quartz crystal unit having an electrode. The QCM sensor can measure slight mass change using a property in which the frequency of the quartz crystal unit is changed (decreased) depending on the mass of the sublimate that is attached to a surface (electrode) of the quartz crystal unit.

A detailed measurement protocol is as follows. The temperature of the hot plate of the sublimate amount measurement apparatus was set to 215° C., the pump flow rate was set to 1 m³/s, and the apparatus was left for first 60 seconds so as to stabilize the apparatus. Immediately, the silicon wafer coated with the resist underlayer film forming composition was placed on the hot plate rapidly from a slide outlet. The sublimate was collected from a time point of 60 seconds to a time point of 180 seconds (for 120 seconds). The resist underlayer film formed on the silicon wafer had a thickness of 0.1 μm.

A flow attachment (detection portion) connecting the QCM sensor of the sublimate amount measurement apparatus to a catching funnel portion was used without a nozzle. A gas flow was poured without being restricted from a flow channel (caliber: 32 mm) of a chamber unit located at a distance of 30 mm from the sensor (quartz crystal unit). The QCM sensor in which a material (AlSi) containing silicon and aluminum as main components is used for an electrode, the diameter of the quartz crystal unit (sensor diameter) is 14 mm, the diameter of the electrode on a surface of the quartz crystal unit is 5 mm, and the resonant frequency is 9 MHz was used.

The obtained frequency change was converted from eigenvalue of the quartz crystal unit used in the measurement into grams, and a relationship between the sublimate amount in one silicon wafer with the resist underlayer film and time course was clarified. Table 1 showed the sublimate amount generated from each of the resist underlayer film forming compositions in Examples 1 to 4 and Comparative Example 2 when the sublimate amount on a time point of 120 seconds in Comparative Example 1 was 1.00. The results showed that the sublimate amounts generated from the resist underlayer film forming compositions in Examples 1 to 4 were smaller than the sublimate amount generated from the composition in Comparative Example 1.

TABLE 1

| | | | Optical parameter | | | | | |
|---|---|---|---|---|---|---|---|---|
| | Solvent | | 193 nm | | 248 nm | | Etching | |
| | resistance | | n | k | n | k | selection | Sublimate |
| | PGME | PGMEA | value | value | value | value | ratio | amount |
| Example 1 | ○ | ○ | 1.82 | 0.31 | 1.66 | 0.00 | 3.14 | 1.00 |
| Example 2 | ○ | ○ | 1.77 | 0.30 | 1.69 | 0.22 | 2.40 | 0.51 |
| Example 3 | ○ | ○ | 1.79 | 0.29 | 1.69 | 0.22 | 2.35 | 0.50 |
| Example 4 | ○ | ○ | 1.77 | 0.29 | 1.70 | 0.22 | 2.31 | 0.69 |
| Comparative | ○ | ○ | 1.63 | 0.28 | 1.83 | 0.61 | 1.40 | 1.00 |
| Example 1 | | | | | | | | |
| Comparative | ○ | ○ | 1.73 | 0.20 | 1.88 | 0.19 | 2.41 | 4.43 |
| Example 2 | | | | | | | | |

The results in Table 1 show that k values at wavelength of 193 nm of the resist underlayer films formed from the resist underlayer film forming compositions in Examples 1 to 4 and Comparative Examples 1 and 2 are larger than 0.1. This shows that the resist underlayer films have an anti-reflection function against the wavelength. Further, the results show that the dry etching rates of the resist underlayer films formed from the resist underlayer film forming compositions in Examples 1 to 4 and Comparative Example 2 are largely higher than the dry etching rate of the photoresist film. On the other hand, the results do not show that the dry etching rate of the resist underlayer film formed from the resist underlayer film forming composition in Comparative Example 1 is largely higher than the dry etching rate of the photoresist film. Moreover, the results show that the sublimate amounts of the resist underlayer films formed from the resist underlayer film forming compositions in Examples 1 to 4 are largely smaller than the sublimate amount of the resist underlayer film formed from the resist underlayer film forming composition in Comparative Example 2. On the other hand, the results show that the sublimate amount of the resist underlayer film formed from the resist underlayer film forming composition in Comparative Example 2 is largely larger than the sublimate amount of the resist underlayer film formed from the resist underlayer film forming composition in Comparative Example 1. These results show that from the resist underlayer film forming compositions in Examples 1 to 4, a resist underlayer film having low sublimation and high dry etching rate may be formed.

[Test of Embeddability (Filling Properties)]

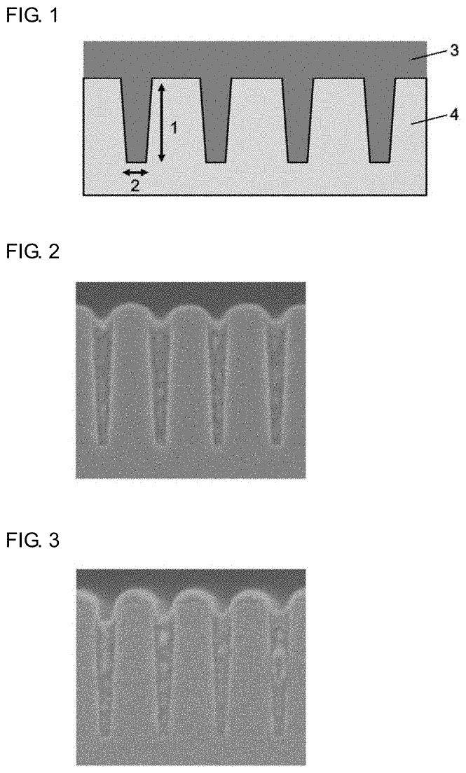

The resist underlayer film forming composition in each of Examples 1 to 4 and Comparative Example 1 was applied to a silicon wafer (hereinafter abbreviated as SiO₂ wafer in the specification) having a plurality of trenches (width: 0.04 μm, depth: 0.3 μm) and an SiO₂ film on a surface by a spinner, and then baked on a hot plate at 215° C. for one minute to form each resist underlayer film (thickness: 0.1 μm). FIG. 1 is a schematic view of a SiO₂ wafer 4 used in this test and a resist underlayer film 3 formed on the wafer. The wafer 4 has trenches in a dense pattern (Dense). The dense pattern is a pattern in which an distance between the center of each trench and the center of each adjacent trench is 3 times the trench width. A depth 1 of the trenches of the SiO₂ wafer 4 shown in FIG. 1 is 0.3 μm and a width 2 of the trenches is 0.04 μm.

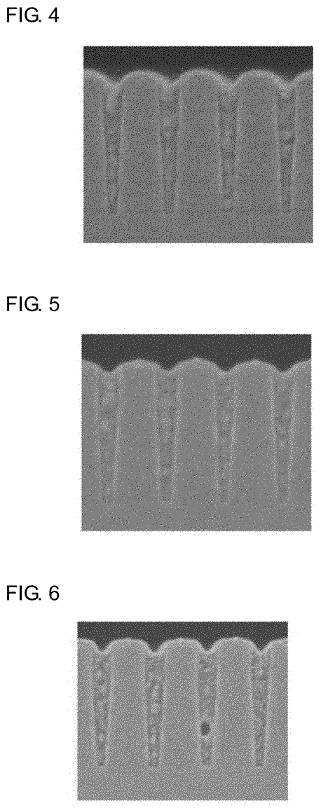

As described above, the resist underlayer film forming composition in each of Examples 1 to 4 and Comparative Example 1 was applied to a SiO₂ wafer and baked to form the resist underlayer film. The cross-sectional shape of the SiO₂ wafer was observed by a scanning electron microscope (SEM). The embeddability of the resist underlayer film forming composition in the trenches of the SiO₂ wafer (filling properties of the resist underlayer film forming composition in the trenches of the SiO₂ wafer) was evaluated. The obtained results are shown in FIG. 2 (Example 1), FIG. 3 (Example 2), FIG. 4 (Example 3), FIG. 5 (Example 4), and FIG. 6 (Comparative Example 1). In the cross-sectional SEM image of the SiO₂ wafer in FIGS. 2 to 5, voids (gaps) in the trenches were not observed, and the trenches were filled with the resist underlayer film and the whole trenches were completely embedded. However, in the cross-sectional SEM image of the SiO₂ wafer in FIG. 6, voids (gaps) in the trenches were observed. These results show that the resist underlayer film forming compositions in Examples 1 to 4 are a material having more excellent embeddability (filling properties) as compared with the resist underlayer film forming composition in Comparative Example 1.

### DESCRIPTION OF SYMBOLS

1 Depth of trench of SiO₂ wafer

2 Width of trench of SiO₂ wafer

3 Resist underlayer film

4 SiO₂ wafer

1. A resist underlayer film forming composition comprising a compound having at least two substituents of the following formula (1): 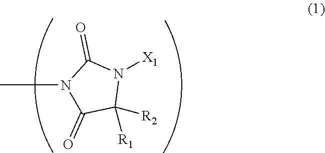
wherein R₁ and R₂ are each independently a hydrogen atom or a methyl group, and X₁ is a C₁₋₃ hydroxyalkyl group or a C₂₋₆ alkyl group having one or two ether bonds in a main chain in a molecule, and a solvent.

2. The resist underlayer film forming composition according to claim 1, wherein the compound is a compound of the following formula (2): 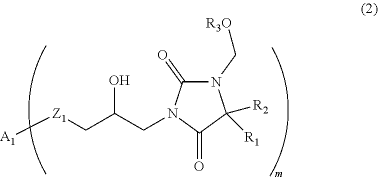
wherein A₁ is a divalent to octavalent aliphatic group or a group having an aromatic or heterocyclic ring, Z₁ is a direct bond, an —O— group, or a —C(═O)O— group, R₁ and R₂ have the same definition as that in formula (1), R₃ is a hydrogen atom, a linear or branched alkyl group having a carbon atom number of 1 to 4, or a C₂₋₅ alkoxyalkyl group, and m is an integer of 2 to 8)2 to 8 having a weight average molecular weight of 300 to 5,000.

3. The resist underlayer film forming composition according to claim 2, wherein in the compound of formula (2), m is an integer of 2 to 4, and A₁ is a divalent, trivalent, or tetravalent aliphatic group or a group having an aromatic or heterocyclic ring.

4. The resist underlayer film forming composition according to claim 2, wherein the compound of formula (2) is a monomeric compound of the following formula (2a): 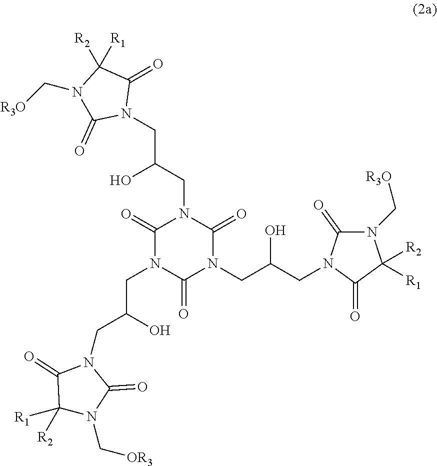
wherein R₁ and R₂ have the same definition as that in formula (1), and R₃ has the same definition as that in formula (2))formula (2).

5. The resist underlayer film forming composition according to claim 2, further comprising a compound of the following formula (3): 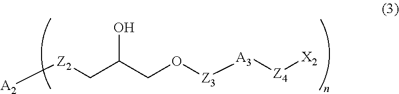
wherein A₂ is a divalent to octavalent aliphatic group or a group having an aromatic or heterocyclic ring, Z₂ is a direct bond, an —O— group, or a —C(═O)O— group, Z₃ and Z₄ are each independently a direct bond or a carbonyl group, A₃ is an arylene group in which at least one hydrogen atom is optionally substituted with hydroxyl group or halogeno group, or a C₁₋₃ alkylene group, X₂ is a hydroxyl group, a cyano group, or a C₁₋₆ alkyl group having one or two oxygen atoms in a main chain, and n is an integer of 2 to 8 in an amount of 1% by mass to 1,000% by mass relative to 100% by mass of the compound of formula (2).

6. The resist underlayer film forming composition according to claim 1, further comprising an additive selected from the group consisting of a cross-linking catalyst, a crosslinkable compound, and a surfactant.

7. The resist underlayer film forming composition according to claim 1, wherein the cross-linking catalyst is a thermal acid generator.

8. A method for forming a photoresist pattern used in manufacturing a semiconductor device comprising steps of:
applying the resist underlayer film forming composition according to claim 1 to a semiconductor substrate having a hole or trench, and baking the semiconductor substrate at 150° C. to 350° C. to form a resist underlayer film;
forming a photoresist layer on the resist underlayer film;
exposing the semiconductor substrate coated with the resist underlayer film and the photoresist layer; and
developing the photoresist layer after exposure.
